(19) United States
(12) Patent Application Publication
(10) Pub. No.: US 2005/0209684 A1
(43) Pub. Date: Sep. 22, 2005

(54) Surgical stent having micro-geometric patterned surface

(76) Inventors: Harold Alexander, Short Hills, NJ (US); John L. Ricci, Middleton, NJ (US)

Correspondence Address: MELVIN K. SILVERMAN, 500 WEST CYPRESS CREEK ROAD, SUITE 500, FT. LAUDERDALE, FL, 33309, US

(21) Appl. No.: 11/071,952

(22) Filed: Mar. 3, 2005

Related U.S. Application Data
(60) Provisional application No. 60/550,130, filed on Mar. 4, 2004.

(51) Int. Cl. A61F 2/06

(52) U.S. Cl. 623/1.15

(57) ABSTRACT

A surgical stent having thereon micro-geometric patterned surface and the method of use for inhibiting smooth muscle cell growth into stent lumen are disclosed. The surgical stent has a generally cylindrical stent frame configured to be implanted into a body lumen, and the stent frame has thereon a micro-geometric patterned surface which includes a multiplicity of microgrooves distributed in a predetermined pattern. Each of the microgrooves has a width in a range of from about 4 to about 40 microns and a depth in a range of from about 4 to about 40 microns. The surgical stent can further include drug wells, and the surgical stent can have a biocompatible chemical compound, such as thrombosis inhibitor or cell growth inhibitor, embedded in the microgrooves or drug wells.

### CROSS REFERENCE TO RELATED APPLICATION

This application claims the benefit under 35 USC 119 (e) of the provisional patent application Ser. No. 60/550,130, filed Mar. 4, 2004, which is hereby incorporated by reference in its entirety.

### FIELD OF THE INVENTION

The present invention relates to a surgical stent for implantation into a body lumen, such as an artery. More specifically, the present invention relates to a surgical stent which has a micro-geometric patterned surface on the stent frame to inhibit smooth muscle cell growth in the stent lumen and to reduce in-stent restenosis.

### BACKGROUND OF THE INVENTION

Surgical stents have long been known which can be surgically implanted into a body lumen, such as an artery, to reinforce, support, repair or otherwise enhance the performance of the lumen. For instance, in cardiovascular surgery it is often desirable to place a stent in a coronary artery at a location where the artery is damaged or is susceptible to collapse. The stent, once in place, reinforces that portion of the artery allowing normal blood flow to occur through the artery. One form of stent which is particularly desirable for implantation in arteries and other body lumens is a cylindrical stent which can be radially expanded from a smaller diameter to a larger diameter. Such radially expandable stents can be inserted into the artery by being located on a catheter and fed internally through the arterial pathways of the patient until the unexpanded stent is located where desired. The catheter is fitted with a balloon or other expansion mechanism which exerts a radial pressure outward on the stent causing the stent to expand radially to a larger diameter. Such expandable stents exhibit sufficient rigidity after being expanded that they will remain expanded after the catheter has been removed.

The balloon-expandable metallic stents make up 99% of the implantable devices used in the treatment of coronary artery disease, and they come in a variety of different configurations to provide optimal performance in various different particular circumstances.

The implanted artery stent keeps coronary arteries open after balloon angioplasty. The stent then allows the normal flow of blood and oxygen to the heart. Stents are also used in other structures such as the esophagus to treat a constriction, the ureters to maintain the drainage of urine from the kidneys, and the bile duct to keep it open.

However, in-stent restenosis remains the major limitations of vascular stenting. Restenosis is the reocclusion, or reclogging, of a coronary artery following a successful intravascular procedure, such as balloon angioplasty or stent placement. It has been shown in the past decade that the rate of in-stent restenosis can be as high as 40%, depending on the designs and materials of the stent, patients, lesions and procedures.

In-stent restenosis is essentially tissue regrowth, the body's overzealous attempt to heal the intima (innermost layer of vessel lining) where it was disturbed by the placement of the coronary artery stent. In response to vascular trauma, growth factors are produced. These growth factors stimulate smooth muscle cells to start dividing, a process known as neointimal hyperplasia. As the smooth muscle cells multiply, they push through the openings in the stent mesh and, over time, cause a narrowing in the stent lumen.

It has been found that the stent geometry, dimensions and stent surface properties appear to highly influence both thromosis and restenosis rates. Next to optimizing stent properties and profile, stent materials and coating have been recently investigated to improve hemocompatibility and tissue compatibility (biocompatibility). This is even more important because it has become clear that treatment of restenosis and especially in-stent restenosis still has poor results, and the best way to diminish these refractory restenotic lesions is their prevention.

All currently available stents are composed of metal. Nearly all balloon-expandable stents in use today are made from 316L stainless steel. This alloy is relatively easy to work with, can be plastically deformed to large expansion ratios without yielding or fatiguing, has low intrinsic elastic recoil, and has a long history of hemocompatibility. Currently, the stents are generally electropolished to a mirror-quality finish, because removal of microscopic roughness appears to decreases platelet adhesion when a stent is exposed to flowing blood in vitro extracorporeal shunt models (Scott et al, Am Heart J. 1995; 129:866-872).

The most recent advance in reducing in-stent restenosis is a drug coated stent, also known as medicated stent, or drug-eluting stent. A drug which inhibits cell growth is coated on the stent surface with thin (5-10μ) elastomeric biostable polymer surface membrane coatings. The most recent designs have the drug filled with bioerodable polymer into drug wells which are embedded in the struts of the stent. Typically, the drug starts to release immediately after implantation. With the drug well design to delay the initial burst release, the release time can be extended to about 20 days.

In April 2003, FDA approved the CYPHER™ sirolimus-eluting coronary stent manufactured by Cordis Corporation, a Johnson & Johnson company, Miami, Fla. From April to October 2003, more than 200,000 patients in the United States were treated with the CYPHER™ stent. It has been reported that the drug-eluting stents have reduced the incidence of in-stent restenosis. However, adverse responses to the drug-eluting stent have also been reported, which led to FDA's issuance of public health notification regarding the CYPHER™ stent in October, 2003. Among the patients treated, there were 290 incidences of sub-acute thrombosis; 60 resulting patient death, and the remainder required medical or surgical intervention. There were also reports of hypersensitivity reactions with symptoms including pain, rash, respiratory alterations, hives, itching, fever, and blood pressure changes.

Based on the above, it is apparent that there remains the need of improving the existing drug-eluting stents, and developing alternative designs and methods for inhibiting smooth muscle cell proliferation to reduce in-stent restenosis.

Smooth muscle cells in blood vessel walls have an elongated morphology and align in the circumferential direction with well-organized structure. It is known that in contrast, smooth muscle cells grown in vitro on smooth surfaces spread randomly on culture surfaces without organized structure, and they do not exhibit elongated morphology.

U.S. Pat. No. 6,419,491 (to Ricci et al) discloses a dental implant with repeating microgeometric surface patterns. Ricci et al have shown that on a surface having alternating micromicrogrooves and ridges with a groove width from 6 to 12 microns, both rat tendon fibroblast (RTF) and rat bone marrow (RBM) cells have elongated colony growth, accelerated in the direction of the microgrooves, and inhibited in the perpendicular direction of the microgrooves. However, with the surface having micromicrogrooves with a groove width of 2 microns, both types of cells bridge the surfaces on the microgrooves resulting cells with different morphologies from those on the 6 to 12 micron surfaces. The results of the observed effects of these microgroove surfaces on overall RBM and RTF cell colony growth were pronounced. All microgrooving surfaces, with different width of the microgrooves, have caused different growth rates in the direction of the microgrooves versus in the direction perpendicular to the microgrooves. More importantly, this results in suppression of overall growth of both cell colonies compared with controls (the same cell colonies grow on a smooth surface). It is also found that the suppression of cell growth differed between cell types.

Furthermore, Thakar et al (Regulation of Vascular Smooth Muscle Cells by Micropatterning, Biochemical and Biophysical Research Communications 307, 883-890, 2003) disclose that smooth muscle cell culture on a micro-patterned matrix decreases smooth muscle cell proliferation rate, stress fiber formation and a-actin expression. Moreover, Thakar et al have found that the smooth muscle cells grown on micro-patterned collagen strips with narrow groove widths (30 microns or less) approach a linear, elongated morphology similar to smooth muscle cell in vivo.

It has also been shown by Chen et al (Geometric Control of Cell Life and Death, Science 276, 1425-1428, 1997) that decreasing cell spreading area on square or circular shaped islands inhibits endothelial cell proliferation and increases apoptosis.

However, the above references do not teach use of a micro-patterned surface on the surgical stents to control or inhibit smooth muscle cell proliferation in the stent lumen.

### BRIEF DESCRIPTION OF THE DRAWINGS

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

FIG. 1 is a partial perspective view showing a portion of an artery stent of the present invention.

FIG. 2 is a partial enlarged schematic view of the artery stent of FIG. 1, showing a multiplicity of alternating microgrooves and ridges on the external surface of the stent frame.

FIGS. 3A to 3H are diagrammatic cross sectional views of various configurations of the microgrooves that can be used on the external surface on the surgical stent.

FIGS. 4 to 5 are diagrammatic plan views illustrating various geometric patterns in which the microgrooves of FIGS. 3A-3H can be arranged.

FIGS. 6 to 13 are also diagrammatic plan views illustrating additional geometric patterns in which the microgrooves of FIGS. 3A-3H can be arranged.

FIG. 14 is a perspective, fragmentary view, part broken away for clarity, of a stent frame surface illustrating a combination of a drug well with the microgrooves.

### SUMMARY OF THE INVENTION

In one aspect, the present invention is directed to a surgical stent which has a micro-geometric patterned surface for inhibiting smooth muscle cell growth into the stent lumen. The surgical stent has a generally cylindrical stent frame configured for implanting into a body lumen, and the stent frame has an external surface having thereon a micro-geometric patterned surface comprising a multiplicity of microgrooves distributed in a pre-determined pattern. Preferably, the micro-geometric patterned surface comprises a multiplicity of alternating microgrooves and ridges. Each of the microgrooves has a width in a range from about 4 to about 40 microns and a depth in a range from about 4 to about 40 microns.

In a further embodiment, the surgical stent further comprises a biocompatible chemical compound on the stent frame. The biocompatible chemical compound can be thrombosis inhibitor, cell growth inhibitor, or combination thereof. The biocompatible chemical compound can be coated on the stent frame, or embedded in the microgrooves. Moreover, the surgical stent further comprises a bioerodable polymer coating the biocompatible chemical compound.

In another embodiment, the surgical stent further comprises a plurality of drug wells and the biocompatible chemical compound embedded in the drug wells. The surgical stent can further comprise a bioerodable polymer coating the embedded biocompatible chemical compound.

The surgical stent of the present invention is an artery stent. It can also be an esophagus stent, or an ureter stent.

In a further aspect, the present invention is directed to a method of inhibiting smooth muscle cell growth into stent lumen of a surgical stent. The method comprises the steps of: providing a surgical stent having a generally cylindrical stent frame, the stent frame having thereon a micro-geometric patterned surface comprising a multiplicity of microgrooves distributed in a pre-determined pattern; and surgically implanting the surgical stent into a body lumen; whereby the multiplicity of microgrooves inhibit smooth muscle cell growth into the stent lumen. The method can further comprise coating the surgical stent with a biocompatible chemical compound including thrombosis inhibitor, cell growth inhibitor, or combination thereof, prior to the implanting the surgical stent into the body lumen. Alternatively, the method comprises embedding the biocompatible chemical compound in the microgrooves prior to the implanting the surgical stent into the body lumen. Additionally, the method further comprises coating the biocompatible chemical compound with a bioerodable polymer, prior to the implanting the surgical stent into the body lumen.

### DETAILED DESCRIPTION OF THE INVENTION

In one embodiment, the present invention provides a surgical stent which has micro-geometric patterned surface for inhibiting smooth muscle cell proliferation in the stent lumen.

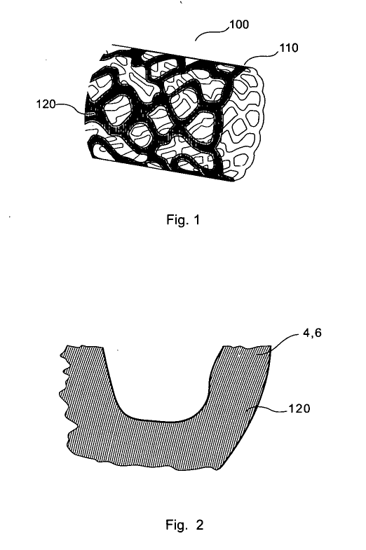

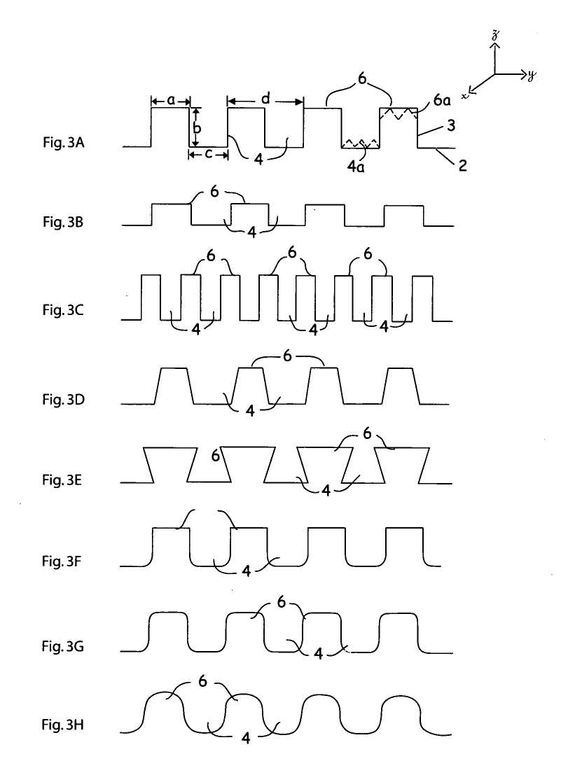

As shown in FIG. 1, the surgical stent 100 has a generally cylindrical stent frame 110 configured to be implanted into a body lumen, such as artery, esophagus stent, or ureter. The surgical stent 100 has an ordered micro-geometric surface pattern comprising a multiplicity of alternating microgrooves 4 and ridges 6 on the external surface 120 of the stent frame 110, as illustrated on the partially enlarged view of the external surface 120 of the stent frame 110 shown in FIG. 2. In FIG. 2, the black lines represent microgrooves 4, and the white areas between the adjacent microgrooves represent ridges 6. The configurations of microgrooves 4 and ridges 6 are described in detail hereinafter.

It should be understood that the stent frame can comprise various structural components and configurations, which include, but are not limited to, spiral articulated slotted tube, sinusoidal pattern, curved sections and interconnected N-links, helically fused sinusoidal elements, sinusoidal ring with elliptical rectangular design, corrugated rings, corrugated ring with curved access links, closed cell having transformable geometry, tendem Architecture and others known in the art. For the purpose of the present invention, the term “stent frame” refers to the formed structure which comprises all major structural components. The term “external surface of the stent frame” used herein refers to the surface of the stem frame that faces the wall of the body lumen. Since the stent frame can comprise more than one components, the external surface of the stent frame includes the external surfaces of various components. Preferably, the microgrooves are placed on the external surface of the major structural components of the stent frame, such as struts, which has a relatively large contact area with the wall of the body lumen.

Some suitable examples of the surgical stent which have the above-described structural features are Cordis Palmaz-Schatz®, Cordis Crown, and Bx Velocity™ by Cordis Corporation, Miami, Fla.; ACS MULTI-LINK®, MULTI-LINK® TETRA and MULTI-LINK® PENTA by Guidant Corporation, Indianapolis, Ind.; NIR® and Express™ by Boston Scientific Corporation, Natick, Mass.; AVE Microstent by Arterial Vascular Engineering, Santa Rosa, Calif.; Inflow by Inflow Dynamics, Munich, Germany; and PURA by Elder, Mumbai, India.

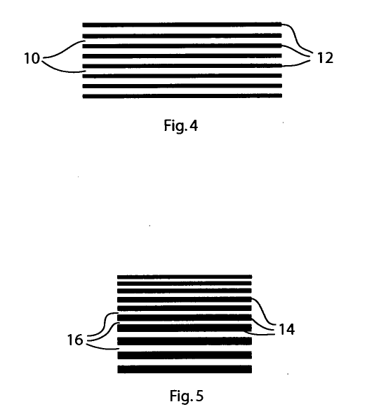

FIGS. 3A to 3H illustrate various suitable configurations of microgrooves 4 and ridges 6, which can be used for forming the ordered micro-geometric surface pattern. Herein, the term “microgroove” refers to a groove having a width and a depth in the order of micrometers, more particularly having a width and a depth less than 50 micrometers.

As shown, each groove has a groove base 2 and a groove wall 3. The dimensions of the microgrooves 4 and ridges 6 are indicated by the letters “a”, “b”, “c” and “d”. These configurations include those having square ridges 6 and square microgrooves 4 (FIG. 3A) where “a”, “b” and “c” are equal and where the spacing (or pitch) “d” between adjacent ridges 6 is twice that of “a”, “b” or “c”. FIGS. 3B and 3C illustrate rectangular configurations formed by microgrooves 4 and ridges 6 where the “b” dimension is not equal to that of “a” and/or “c”.

FIGS. 3D and 3E illustrate trapozidal configurations formed by microgrooves 4 and ridges 6 where the angles formed by “b” and “c” can be either greater than 90° as shown in FIG. 3D or less than 90° as shown in FIG. 3E. As shown in the above-configurations, each groove defines, in radial cross-section thereof, a relationship of the groove base 2 to the grove wall 3, which is in a range from about 60 degree to about 120 degree.

In FIG. 3F, the corners formed by the intersection of dimensions “b” and “c” have been rounded and in FIG. 3G, these corners as well as the corners formed by the intersection of dimensions “a” and “b” have been rounded. These rounded corners can range from arcs of only a few degrees to arcs where consecutive microgrooves 4 and ridges 6 approach the configuration of a sine curve as shown in FIG. 3H.

In all of these configurations, either the planar surface of the ridge 6; i.e., the “a” dimension, or the planar surface of the groove 4; i.e., the “c” dimension, or both can be corrugated as shown by dotted lines at 6a and 4a in FIG. 3A.

In the microgroove configurations illustrated in FIGS. 3A to 3H, the dimension of “c”, i.e., the width of the groove, can be from about 1.5 μm to about 50 μm, preferably from about 4 μm to about 40 μm, and more preferably from about 6 μm to about 28 μm. In the trapozidal configurations as shown in FIGS. 3D and 3E, the width of the groove can be defined at the width at the half height of the groove. The dimension of “a”, i.e., the width of the ridge, can be equal or different from “c” depending on the design needs. The dimension of “b”, i.e., the depth of the groove, should be similar to “c” for the purpose of inhibiting smooth muscle cell proliferation.

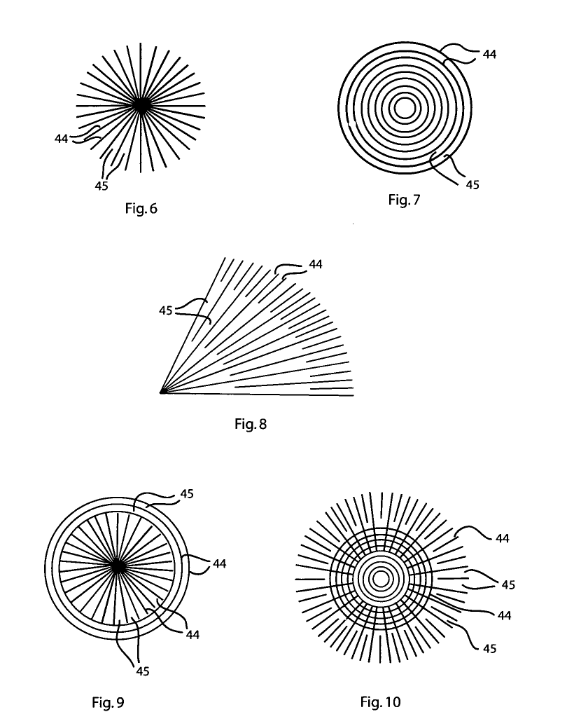

The microgrooves shown in FIGS. 3A-3H can be arranged in various geometric patterns in different embodiments of the present invention, as illustrated in FIG. 4 to FIG. 13. More particularly, with reference to FIG. 4, the microgrooves can be in the form of an infinite repeating pattern of alternating microgrooves 12 and ridges 10. In the embodiment shown in FIG. 5, the microgrooves 14 and ridges 16 increase (or decrease) in width in the direction in perpendicular to the longitudinal axis of the microgrooves.

In a preferred embodiment of the present invention, the co-parallel linear microgrooves 4, as shown in FIG. 2, have a substantially equal width, and the ridges 6 also have a substantial equal width to the microgrooves 4. In the embodiment shown in FIG. 2, the microgrooves are made on the external surface of the stent frame in the circumferential direction of the stent frame, which resembles the alignment of the native smooth muscle cells inside the blood vessel walls. Alternatively, the microgrooves can be aligned in parallel to the longitudinal axis of the stent frame.

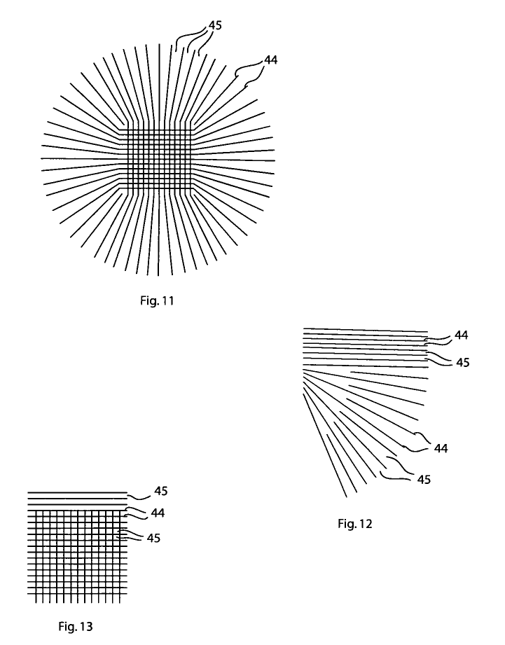

Furthermore, FIGS. 6 to 13 show additional geometric patterns that the microgrooves of FIGS. 3A to 3H can be arranged in the form of unidirectional, arcuate and radial patterns as well as combinations thereof. As shown, these geometric patterns include radiating patterns (FIG. 6); concentric circular patterns (FIG. 7); radiating fan patterns (FIG. 8); radiating/concentric circular patterns (FIG. 9); radiating pattern intersecting concentric circular pattern (FIG. 10); an intersecting pattern surrounded by a radiating pattern (FIG. 11); a combination radiating fan pattern and parallel pattern (FIG. 12); and, a combination intersecting pattern and parallel pattern (FIG. 13). In all these figures, the black lines indicate the microgrooves (44), and the white areas between the adjacent microgrooves indicate the ridges (45).

From the embodiments illustrated in FIGS. 3A to 3H, FIGS. 4 to 5 and FIGS. 6 to 13, it can be appreciated that surgical stents can be provided with micro-geometric patterned surfaces having a multitude of geometric patterns, configurations and cross sections to select from for particular stent applications.

The above-described micro-geometric patterned surfaces can be produced on the surface of the stent frame by laser based technologies known in the art, such as the instrument and methodology illustrated in details in U.S. Pat. Nos. 5,645,740 and 5,607,607, which are herein incorporated by reference in their entirety. Preferably, computerized laser ablation techniques can be used to produce the micro-geometric patterned surfaces.

The above-described micro-geometric patterned surfaces produced on the external surface of the stent frame can be utilized to inhibit smooth muscle cell proliferation in the stent lumen. The effectiveness in suppression of overall cell growth on a cell culture surface having the above-described micro-geometric patterns have been described in U.S. Pat. Nos. 5,645,740, 5,607,607 and 6,419,491, which are herein incorporated by reference in their entirety.

More specifically, as described in U.S. Pat. No. 5,645,740, using a titanium oxide surface with the micro-geometric patterns shown in Table 1, a substantial suppression of rat tendon fibroblast (RTF) cell growth was observed in comparison with the control which grew the same type of cells on a flat smooth surface.

TABLE 1

| | |
|---|---|
| | Actual Dimension (μm) |
| Configuration | (a × c × b) |

| | | |
|---|---|---|
| 2 | μm | 1.80 × 1.75 × 1.75 |
| 4 | μm | 3.50 × 3.50 × 3.50 |
| 6 | μm | 3.50 × 3.50 × 3.50 |
| 8 | μm | 8.00 × 7.75 × 7.50 |
| 12 | μm | 12.00 × 11.50 × 7.5 |

Note:
To simplify nomenclature, the configuration used in these studies are referred to as 2 μm (a = 1.80 μm), 4 μm (a = 3.50 μm), 6 μm (a = 6.50 μm), 8 μm (a = 8.00 μm), and 12 μm (a = 12.00 μm).

The micro-geometric patterned surfaces were observed to result in elongated colony growth in the direction along the longitudinal axis (also referred to as x-axis) of the microgrooves and inhibition of cell growth in the direction perpendicular to the longitudinal axis (also referred to as y-axis) of the microgrooves. On an individual cell level, the cells had elongated morphology and appeared to be “channelled” along the microgrooves, as compared with control culture where outgrowing cells move randomly on flat surfaces. The most efficient “channelling” was observed on the 6 μm and 8 μm surfaces. On these surfaces, the rat tendon fibroblast cells were observed to attach and orient within the microgrooves. This rendered almost no growth in the y-axis on these surfaces.

On smaller micro-geometries, a different effect was observed. The RTF cells bridged the surfaces on the 2 μm microgrooves resulting in cells with different morphologies from those on the 6, 8, and 12 μm surfaces. These cells were wide and flattened and were not well oriented. On the 4 μm microgrooves, the RTF cells showed mixed morphologies, with most cells aligned and elongated but not fully attached within the microgrooves. This resulted in appreciable growth of the RTF cells in the y-axis on the 2 and 4 μm surfaces. At the other end, limited y-axis growth was also observed when the RTF cells were grown on the 12 μm surfaces.

The results of the observed effects of these surfaces on overall RTF cell colony growth were pronounced. All micro-geometric patterned surfaces tested caused varying but significant increases in x-axis growth compared to the diameter increase of the controls, and varying but pronounced inhibition of y-axis growth. More importantly, this resulted in suppression of overall growth of the RTF cell colony compared with the control. It is also shown that the suppression of cell growth differed between different types of cells.

It is important to point out that the RTF cells grown on the micro-geometric patterned surfaces with 6 to 12 μm microgrooves had elongated morphology, which is the morphology of the smooth muscle cells in the native blood vessel walls. Furthermore, the native smooth muscle cells align in the circumferential direction with well-organized structure. Although the exact mechanism of the effect of cell morphology on smooth muscle cell proliferation is not known, it could be due to different tension distribution inside the cells (S. Hung, D. E. Ingber, The structural and mechanical complexity of cell-growth control, Nat. Cell Biol., 1 (1999) 1 E131-138).

Therefore, incorporating these micro-geometric patterns on to the external surface of the stent frame inhibits the smooth muscle cell proliferation in the stent lumen. As stated previously, the stent frame in the context of the present invention includes all major structural components of the stent.

In a further embodiment, the micro-geometric patterns of the present invention can be combined with the drug eluting stents. In one embodiment, a biocompatible chemical compound is coated on the surgical stent using the existing method known in the art. One suitable example is the ultrasonic spray method developed by Sono-Tek Corporation, Milton, N.Y. The biocompatible chemical compound can be a thrombosis inhibitor, a cell growth inhibitor, or combination thereof. Preferably, the biocompatible chemical compound is coated with bioerodable polymers for providing time release of the chemical compound. The existing bioerodable polymers used in the drug eluting stents can be used for the purpose of the present invention.

In another embodiment, the biocompatible chemical compound is embedded in the microgrooves of the stent frame, and preferably further coated with the coated with bioerodable polymers.

In yet a further embodiment, the micro-geometric patterns of the present invention can be combined with the existing drug well design on the surface of the stent frame, thereby providing both chemical and geometric inhibitions of the smooth muscle cell proliferation at the same time. The drug well can be either on the external surface or internal surface (facing the inside of the stent lumen). In this embodiment, the micro-geometric patterns and the drug wells are so arranged that the drug wells do not substantially interfere with the microgrooves.

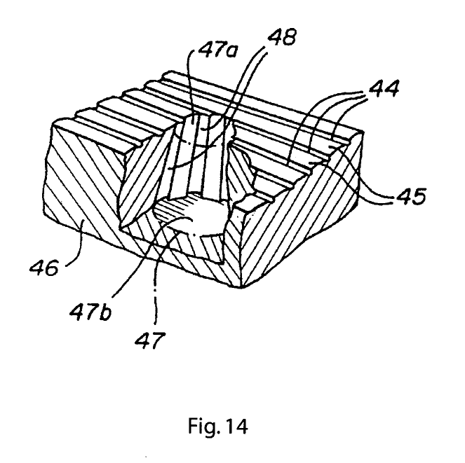

FIG. 14 illustrated a combination of microgrooves with a drug well. As shown, microgrooves 44 and ridges 45 are formed in the external surface of a strut of a stent, which extend and connect to a drug well 47. The drug well has an open top 47a and a closed bottom 47b. While the drug well 47 can be various geometric configuration, it is here shown in the form of a frustoconical shape, the circumference of open top 47a being smaller than the circumference of closed bottom 47b. Optionally, the circumferential wall of drug well 47 can have a plurality of spaced, longitudinal microgrooves 48 formed therein. It is noted that drawing in FIG. 14 is exaggerated for the purpose of illustration.

The structures and method of making drug wells on surgical stents are known in the art. One suitable example is the artery stent, which has a plurality of small wells that serve as drug reservoirs, described in European Patent No. EP 0 706 376, which is hereby incorporated by reference in its entirety. Another suitable example is the Conor stent, made by Conor MedSystems, Inc., Menlo Park, Calif.

With anyone of the above-described configurations, the micro-geometric patterned drug eluting stents have double benefit of the chemical inhibition and geometric inhibition on the proliferation of smooth muscle cells. It should be understood that the current drug eluting stent releases its surface coated drug in a short period of time, i.e., in days. Therefore, after the complete release of the coated drug, there is no mechanism to prevent growth of the smooth muscle cell into the stent lumen. With the micro-geometric patterned drug eluting stent of the present invention, the patient not only can be benefited by an immediate chemical inhibition of thrombosis and restenosis caused by the surgical disturbances, the patient can also have a long term benefit of geometric inhibition provided by the micro-geometric patterned surface on the surgical stent. Furthermore, because of the presence of the geometric inhibition mechanism, one can reduce the amount of drug coated on the stent surface, which can reduce potential negative response of the patient to the drug.

In a further aspect, the present invention provides a method of inhibiting smooth muscle cell proliferation upon stent implantation. The method comprises surgically implanting a surgical stent into a body lumen, wherein the surgical stent has one or more above-described micro-geometric patterns on the external surface of the stent frame, whereby the micro-geometric patterned surface inhibits smooth muscle cell growth into a stent lumen. The method further comprises coating the stent frame or embedding the microgrooves or the drug wells, with the biocompatible chemical compound, and further coating the biocompatible chemical compound with a bioerodable polymer, as described above.

As described previously, an improved surgical stent which reduces in-stent restenosis has been a long felt need in the medical field. The present invention is the first to provide a geometric inhibition mechanism by incorporating micro-geometric patterns on to the stent surface, thereby inhibiting smooth muscle cell growth into the stent lumen.

While the present invention has been described in detail and pictorially shown in the accompanying drawings, these should not be construed as limitations on the scope of the present invention, but rather as an exemplification of preferred embodiments thereof. It will be apparent, however, that various modifications and changes can be made within the spirit and the scope of this invention as described in the above specification and defined in the appended claims and their legal equivalents.

1. A surgical stent having a generally cylindrical stent frame configured for implanting into a body lumen, said stent frame having an external surface; said external surface having thereon a micro-geometric patterned surface comprising a multiplicity of microgrooves distributed in a pre-determined pattern.

2. The surgical stent of claim 1 wherein each of said microgrooves having a width in a range from about 4 to about 40 microns (micrometers) and a depth in a range from about 4 to about 40 microns.

3. The surgical stent of claim 2, wherein each of said microgrooves has a groove base and a groove wall, each groove defining, in radial cross-section thereof, a relationship of said groove base to said groove wall, which is from about 60 degree to about 120 degree.

4. The surgical stent of claim 2 further comprising a biocompatible chemical compound on said stent frame; said biocompatible chemical compound being one selected from the group consisting of thrombosis inhibitor, cell growth inhibitor and combination thereof.

5. The surgical stent of claim 4, wherein said biocompatible chemical compound are coated on said stent frame.

6. The surgical stent of claim 4, wherein said biocompatible chemical compound are embedded in said microgrooves.

7. The surgical stent of claim 4 further comprising a bioerodable polymer coating said biocompatible chemical compound.

8. The surgical stent of claim 1 further comprising a plurality of drug wells and a biocompatible chemical compound embedded in said drug wells.

9. The surgical stent of claim 8, wherein said biocompatible chemical compound is one selected from the group consisting of thrombosis inhibitor, cell growth inhibitor and combination thereof.

10. The surgical stent of claim 8 further comprising a bioerodable polymer coating said biocompatible chemical compound.

11. The surgical stent of claim 1 is an artery stent, an esophagus stent, or an ureter stent.

12. A surgical stent having a generally cylindrical stent frame configured for implanting into a body lumen, said stent frame having an external surface; said external surface having thereon a micro-geometric patterned surface comprising a multiplicity of alternating microgrooves and ridges.

13. The surgical stent of claim 12, wherein each of said microgrooves having a width in a range from about 4 to about 40 microns and a depth in a range from about 4 to about 40 microns.

14. The surgical stent of claim 12, wherein said multiplicity of alternating microgrooves and ridges having a substantially same width and a substantially same depth.

15. The surgical stent of claim 12 further comprising a biocompatible chemical compound on said stent frame; said biocompatible chemical compound being one selected from the group consisting of thrombosis inhibitor, cell growth inhibitor and combination thereof.

16. The surgical stent of claim 12 is an artery stent, an esophagus stent, or an ureter stent.

17. A method of inhibiting smooth muscle cell growth into stent lumen of a surgical stent comprising the steps of:
(a) providing a surgical stent having a generally cylindrical stent frame, said stent frame having thereon a micro-geometric patterned surface comprising a multiplicity of microgrooves distributed in a pre-determined pattern; and
(b) surgically implanting said surgical stent into a body lumen;
whereby said multiplicity of microgrooves inhibit smooth muscle cell growth into said stent lumen.

18. The method of claim 17 further comprising coating said surgical stent with a biocompatible chemical compound prior to said implanting said surgical stent into said body lumen; said biocompatible chemical compound being selected from the group consisting of thrombosis inhibitor, cell growth inhibitor and combination thereof.

19. The method of claim 17 further comprising embedding a biocompatible chemical compound in said microgrooves prior to said implanting said surgical stent into said body lumen; said biocompatible chemical compound being selected from the group consisting of thrombosis inhibitor, cell growth inhibitor and combination thereof.

20. The method of claim 19 further comprising coating said biocompatible chemical compound with a bioerodable polymer, prior to said implanting said surgical stent into said body lumen.
